(19) United States
(12) Patent Application Publication
(10) Pub. No.: US 2016/0106478 A1
(43) Pub. Date: Apr. 21, 2016

(54) SURGICAL SYSTEM AND METHOD

(71) Applicant: Warsaw Orthopedic, Inc., Warsaw, IN (US)

(72) Inventors: Joshua W. Simpson, Collierville, TN (US); William Alan Rezach, Atoka, TN (US); Kenneth R. Barsch, Cordova, TN (US); Gary S. Lindemann, Collierville, TN (US); Jason May, Cordova, TN (US)

(21) Appl. No.: 14/518,579

(22) Filed: Oct. 20, 2014

(51) Int. Cl. A61B 17/70; A61B 17/82

(52) U.S. Cl. CPC A61B 17/7053; A61B 17/82; A61B 2017/564

(57) ABSTRACT

A spinal construct comprises a longitudinal member extending between a first end and a second end. A reduction member is connected with the second end and defines at least one opening configured for disposal of the longitudinal member. The reduction member includes an outer surface engageable with a spinal implant and is translatable relative to the longitudinal member. Systems and methods are disclosed.

### TECHNICAL FIELD

The present disclosure generally relates to medical devices for the treatment of musculoskeletal disorders, and more particularly to a surgical system and method for correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Nonsurgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, bone screws and sub-laminar wire, for stabilization of a treated section of a spine. This disclosure describes an improvement over these prior art technologies.

### SUMMARY

In one embodiment, a spinal construct comprises a longitudinal member extending between a first end and a second end. A reduction member is connected with the second end and defines at least one opening configured for disposal of the longitudinal member. The reduction member includes an outer surface engageable with a spinal implant and is translatable relative to the longitudinal member. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of one embodiment of components of a surgical system in accordance with the principles of the present disclosure;

FIG. 2 is a break away view of the components of the system shown in FIG. 1;

FIG. 3 is a break away view of one embodiment of components of a surgical system in accordance with the principles of the present disclosure;

FIG. 4 is a break away view of one embodiment of components of a surgical system in accordance with the principles of the present disclosure;

FIG. 5 is a break away view of the components shown in FIG. 1;

FIG. 6 is a break away perspective view of one embodiment of components of a surgical system in accordance with the principles of the present disclosure disposed with vertebrae;

FIG. 7 is a break away perspective view of the components and vertebrae shown in FIG. 6; and

FIG. 8 is a perspective view of one embodiment of components of a surgical system in accordance with the principles of the present disclosure disposed with vertebrae.

### DETAILED DESCRIPTION

The exemplary embodiments of a surgical system and related methods of use are discussed in terms of medical devices for the treatment of musculoskeletal disorders and more particularly, in terms of a surgical system and method for correction of a spine disorder. In some embodiments, the surgical system includes a surgical system that may be employed in applications for correction of deformities, such as scoliosis and kyphosis.

In some embodiments, the surgical system includes a spinal construct having a longitudinal element, such as, for example, a tether and a reduction member, such as, for example, a crimp. In one embodiment, the spinal construct includes a crimp configured as a buckle. In some embodiments, the surgical system utilizes minimal hardware to simplify cost and a surgical procedure.

In some embodiments, the surgical system includes a crimp that includes two openings to receive ends of a tether. In some embodiments, the crimp is pre-assembled with the tether. In some embodiments, the crimp is attached with the tether in situ. In some embodiments, the tether includes a free end configured to be wrapped around laminae and attached to the crimp for tightening around an implant, such as, for example, a spinal rod. In one embodiment, the crimp is configured to fix the tether to the spinal rod. In some embodiments, an excess portion of the tether is trimmed.

In some embodiments, the surgical system includes a spinal construct having a portion of the tether with a greater width in the area where the tether connects with the laminae to spread out the force over a larger area to reduce pressure on the tether. In some embodiments, the crimp may include only a single opening.

In some embodiments, the surgical system includes a spinal construct that may be employed with various methods to crimp a reduction member to fix a tether to a spinal rod. In some embodiments, the crimp includes teeth disposed with an inner surface thereof. In one embodiment, a reduction member is positioned adjacent a spinal rod such that the spinal rod and a tether are held next to each other by the reduction member and not fixed to each other.

In some embodiments, the surgical system includes a doubled posterior sublaminar tether. In some embodiments, the surgical system includes a spinal construct having a sublaminar tether that is folded to provide more surface area under laminae and facilitate connection with a pre-attached crimp.

In some embodiments, the surgical system includes a spinal construct having a tether that is folded in half and the ends are placed on top of each other to facilitate disposal of other features of the system over the ends of the tether.

In some embodiments, the surgical system includes a spinal construct having a crimp disposed about a center of the tether prior to folding the tether. In one embodiment, the tether is folded around the crimp and the crimp is fixed in place by stitching, ultrasonic welding and/or adhesive. In one embodiment, the crimp includes two channels that allow for passage of the tether. In some embodiments, the crimp can have one channel or multiple channels.

In some embodiments, the surgical system includes a spinal construct having a tether configured to be passed under laminae and then passed through the reduction member. The crimp is compressed in such a way that it would hold the rod in place by locking the tether and not allowing it to travel in the reverse direction.

In some embodiments, the surgical system includes a spinal construct configured to be crimped by a crimple having offset teeth that pinch the crimp in the center. In one embodiment, the crimp includes two sets of teeth configured to pinch the crimp at either end. In one embodiment, the crimp includes three offset teeth configured to cause the tether to follow a torturous path, such as, for example, twisting and/or a plurality curves, through the crimp. In some embodiments, the surgical system includes a spinal construct having a crimp with teeth configured to pierce through the crimp and into the tether.

In some embodiments, the surgical system includes a spinal construct having a tether that is crimped to a connector to secure the tether to the connector and the spinal rod, which are disposed adjacent vertebrae. In some embodiments, this configuration allows the tether to have a greater axial grip.

In some embodiments, the surgical system is used with surgical navigation, such as, for example, fluoroscope or image guidance. In some embodiments, the presently disclosed systems and methods reduce operating time for a surgical procedure and reduce radiation exposure due to fluoroscope or image guidance, for example, by eliminating procedural steps and patient repositioning by implanting system components in one body position.

In one embodiment, one or all of the components of the surgical system are disposable, peel-pack, pre-packed sterile devices. One or all of the components of the surgical system may be reusable. The surgical system may be configured as a kit with multiple sized and configured components.

In one embodiment, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In one embodiment, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a.” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

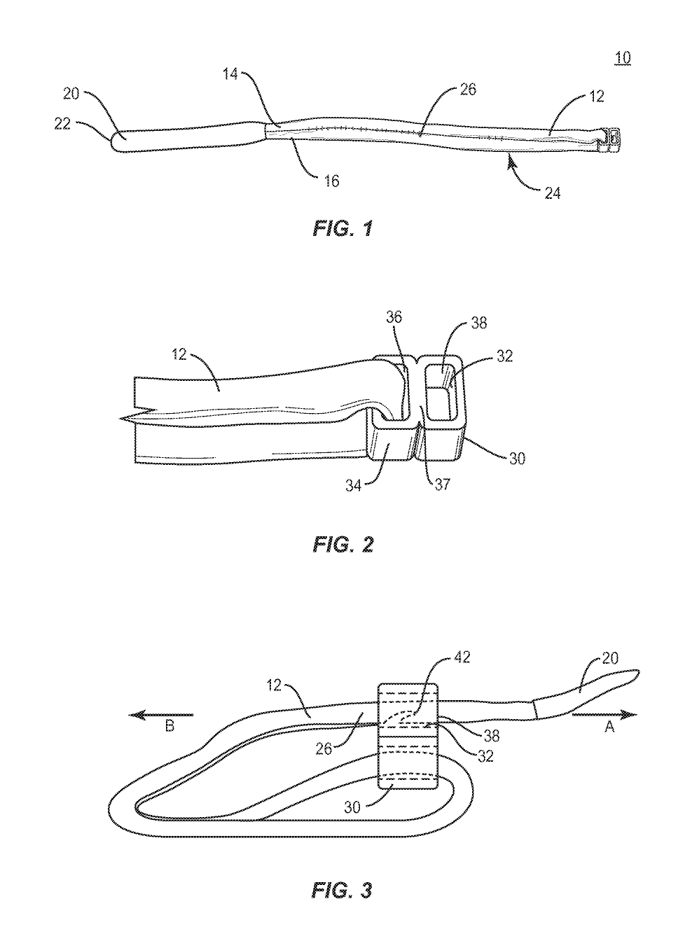

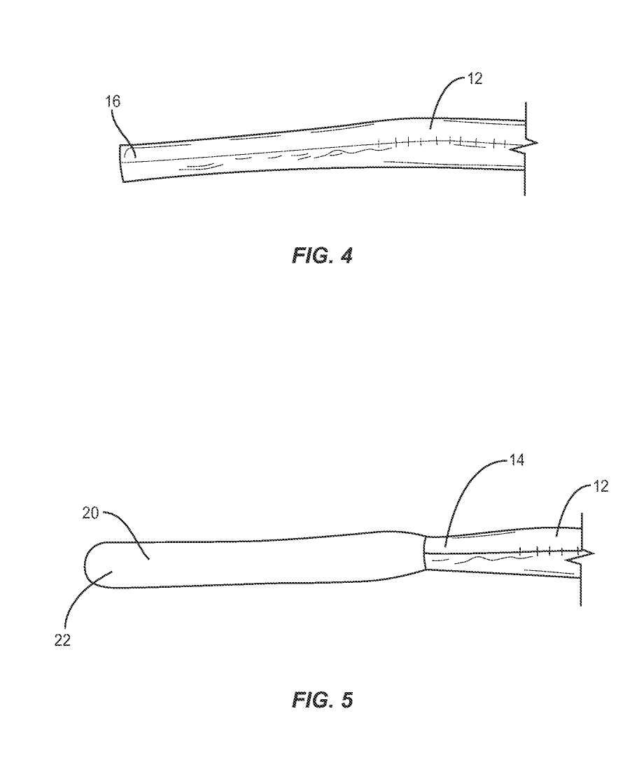

The following discussion includes a description of a surgical system and related methods of employing the surgical system in accordance with the principles of the present disclosure. Alternate embodiments are also disclosed. Reference is made in detail to exemplary embodiments of the present disclosure, which are illustrated in the accompanying figures. Turning to FIGS. 1-5, there are illustrated components of a surgical system, such as, for example, spinal correction system 10.

The components of spinal correction system 10 can be fabricated from biologically acceptable materials suitable for medical applications, including metals, synthetic polymers, ceramics and bone material and/or their composites. For example, the components of spinal correction system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TOP), HA-TCP, calcium sulfate, or other resorbable polymers such as polyaetide, polyglycolide, polytyrosine carbonate, polycaropiaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal correction system 10 may have material composites, including the above materials, to achieve various desired characteristics such as strength, rigidity, elasticity, compliance, biomechanical performance, durability and radiolucency or imaging preference. The components of spinal correction system 10, individually or collectively, may also be fabricated from a heterogeneous material such as a combination of two or more of the above-described materials. The components of spinal correction system 10 may be monolithically formed, integrally connected or include fastening elements and/or instruments, as described herein.

Spinal correction system 10 comprises a spinal construct that includes a longitudinal member, such as, for example, a tether 12. Tether 12 is a flexible longitudinal element that extends between an end 14 and an end 16. End 14 includes a pliable lead, such as, for example, a paddle 20. In one embodiment, paddle 20 includes a blunt tip 22 configured to prevent damage to selected tissue, such as, for example, surrounding tissue and/or nerves adjacent to a surgical site. In one embodiment, paddle 20 connects end 14 with end 16.

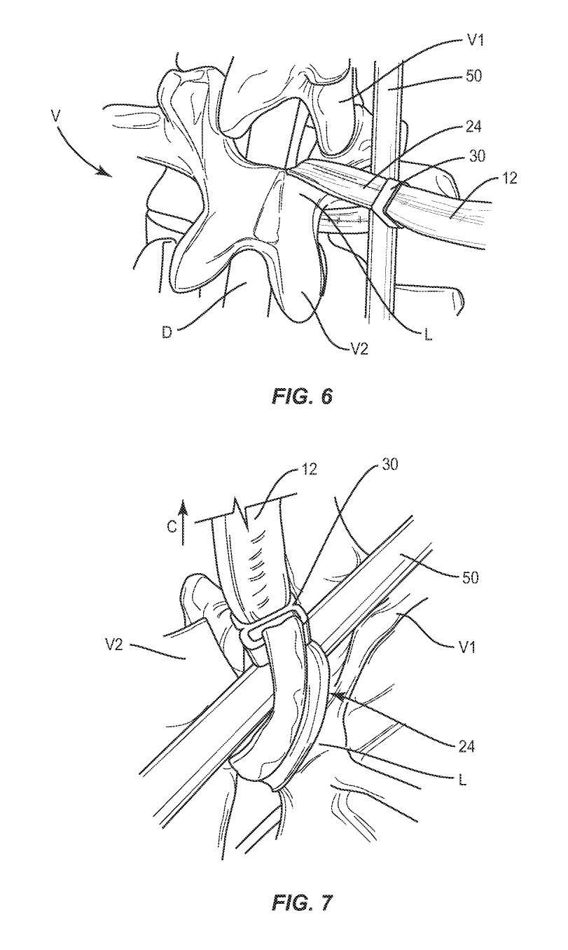

Tether 12 is configured for engagement with a reduction member, such as, for example, a crimp 30, as described herein. In some embodiments, end 14 and end 16 form a loop 24 configured to surround all or a portion of tissue, such as, for example, laminae and/or a spinal implant, such as, for example, a spinal rod 50, as described herein. Tether 12 includes a surface 26 that engages spinal rod 50 and/or tissue, such as, for example, laminae L (FIGS. 6 and 7). In some embodiments, surface 26 includes a larger diameter to spread a tensioned force over a larger area to reduce pressure on tether 12. In one embodiment, tether 12 is folded such that tether 12 is doubled over upon itself to form surface 26. In this configuration, tether 12 can be pre-attached to a reduction member, for example, crimp 30, as described herein.

Tether 12 is configured for tensioning about a targeted portion of an anatomy of a body for attachment of tether 12 with the targeted portion of the anatomy, as described herein. In some embodiments, tether 12 may be manipulated manually and/or with a surgical tensioning instrument. In some embodiments, the targeted portion of the anatomy may include laminae, transverse process and/or pedicle regions of a vertebral level. In some embodiments, spinal correction system 10 may include one or a plurality of tethers 12, each tether being configured for disposal about a single and separate vertebral level. In some embodiments, a single vertebral level may include one or a plurality of tethers 12. In some embodiments, the longitudinal member comprises a rod.

Tether 12 has a flexible configuration and may be fabricated from materials, such as, for example, fabric, silicone, polyurethane, silicone-polyurethane copolymers, polymeric rubbers, polyolefin rubbers, elastomers, rubbers, thermoplastic elastomers, thermoset elastomers and elastomeric composites. In one embodiment, the flexibility of tether 12 includes movement in a lateral or side to side direction and prevents expanding and/or extension in an axial direction upon tensioning and attachment with a targeted portion of the anatomy. In some embodiments, all or only a portion of tether 12 may have a semi-rigid, rigid or elastic configuration, and/or have elastic properties, similar to the material examples described above, such that tether 12 provides a selective amount of expansion and/or extension in an axial direction. In some embodiments, tether 12 may be compressible in an axial direction. Tether 12 can include a plurality of separately attachable or connectable portions or sections, such as bands or loops, or may be monolithically formed as a single continuous element.

Tether 12 can have a uniform thickness/diameter. In some embodiments, tether 12 may have various surface configurations, such as, for example, smooth and/or surface configurations to enhance fixation, such as, for example, rough, arcuate, undulating, porous, semi-porous, dimpled, polished and/or textured. In some embodiments, the thickness defined by tether 12 may be uniformly increasing or decreasing, or have alternate diameter dimensions along its length. In some embodiments, tether 12 may have various cross section configurations, such as, for example, oval, oblong, triangular, rectangular, square, polygonal, irregular, uniform, non-uniform, variable and/or tapered. In some embodiments, the surface of tether 12 may include engaging structures, such as, for example, barbs, raised elements and/or spikes to facilitate engagement with tissue of the targeted anatomy.

In some embodiments, tether 12 may have various lengths. In some embodiments, tether 12 may be braided, such as a rope, or include a plurality elongated elements to provide a predetermined force resistance. In some embodiments, tether 12 may be made from autograft and/or allograft, and be configured for resorbable or degradable applications. In one embodiment, tether 12 is a cadaver tendon. In one embodiment, tether 12 is a tendon that may be harvested, for example, from a patient or donor. In some embodiments, a tendon harvested from a patient may be affixed in remote locations with the patient's body.

Crimp 30 includes an inner surface 32 and an outer surface 34. Surface 32 defines openings 36, 38 for disposal and slidable translation of tether 12. Openings 36, 38 are separated by a wall 37. In some embodiments, surface 32 can define one or more openings for disposal and slidable translation of tether 12. In some embodiments, opening 36 and/or opening 38 may have various cross section configurations, such as, for example, oval oblong, triangular, rectangular, square, polygonal, irregular, uniform, non-uniform, variable and/or tapered. In some embodiments, opening 36 and/or opening 38 may be relatively disposed in a side by side, irregular, uniform, non-uniform, offset and/or staggered orientation or arrangement. In one embodiment, paddle 20 includes connected ends 14, 16 and is passed through opening 36 to pass tether 12 around laminae to form loop 24 around the laminae. Paddle 20 is then passed through opening 38 to close loop 24 of tether 12, which may also be disposed about spinal rod 50, as described herein.

Surface 34 is configured for engagement with a spinal implant, for example, spinal rod 50. Surface 34 is manipulated and/or engaged with a surgical instrument to abut spinal rod 50 such that crimp 30 translates spinal rod 50 to a selected location, such as, for example, a surgical site. In some embodiments, surface 34 may have various surface configurations, such as, for example, rough, threaded for connection with surgical instruments, arcuate, undulating, dimpled, polished and/or textured.

In some embodiments, tether 12 is slidably disposed with opening 36 and/or opening 38, with or without formation of loop 24, such that crimp 30 translates relative to tether 12 between a first or initial position such that tether 12 is loose, slack or includes a lower degree of tension and a second position such that surface 34 abuts spinal rod 50 to apply force to spinal rod 50 for fixation at a surgical site. In some embodiments, in the second position, crimp 30 fixes spinal rod 50 at a surgical site and tether 12 is taught or includes a greater degree of tension relative to the first or initial position.

Crimp 30 is translatable in a first direction and/or in a second opposing direction, as described herein, relative to tether 12 such that crimp 30 translates along tether 12 to drive surface 34 into engagement with spinal rod 50. In some embodiments, as crimp 30 translates along tether 12 and drives spinal rod 50 to the second position at the surgical site, a tensioning force increases in tether 12 causing spinal rod 50 to move adjacent vertebrae. In some embodiments, the tension of tether 12 is selectively increased to selectively fix spinal rod 50 with the vertebrae without the use of tissue penetrating bone fasteners. In some embodiments, in the second position, crimp 30 is deformed, via surgical instrument or manipulation, into engagement with tether 12 such that surface 32 is fixed with surface 26 to prevent translation of crimp 30 relative to tether 12. In some embodiments, in the second position, deformation of crimp 30 includes plastic deformation of surface 32 to reduce the size of opening 36 and/or opening 38, crushing of crimp 30 and/or crimpling. In some embodiments, in the second position, crimp 30 is fixed with tether 12 to prevent translation of crimp 30 relative to tether 12 via tapered openings 36, 38, stitching, ultrasonic welding and/or adhesive.

In one embodiment, as shown in FIG. 3, crimp 30 includes at least one penetrating element, such as, for example, a tooth 42 having a tip oriented in a first direction, as shown by arrow A, and engageable with tether 12 to resist and/or prevent movement of tether 12 in a second opposing direction, as shown by arrow B. As tether 12 is moved in the first direction, tooth 42 extends from surface 32 into opening 38 at an angular orientation such that tether 12 slides over tooth 42 and tooth 42 allows movement, in the direction shown by arrow A. As tether 12 is moved in the second direction, tooth 42 extends from surface 32 to penetrate surface 26 to resist and/or prevent movement of crimp 30 relative to tether 12, in the direction shown by arrow B. In some embodiments, one or a plurality of teeth 42 may be disposed at alternate orientations, relative to surface 32, such as, for example, transverse, and/or other angular orientations such as acute or obtuse, co-axial and/or may be offset or staggered. In some embodiments, crimp 30 may include one or a plurality of penetrating elements. In some embodiments, tooth 42 can be variously configured, such as, for example, nails, serrated, textured, staggered, uneven, undulating, smooth, barbs and/or raised elements.

In some embodiments, spinal rod 50 includes a cylindrical cross section configuration. In some embodiments, spinal rod 50 extends along one or a plurality of vertebra, as described herein. In some embodiments, spinal correction system 10 may include one or a plurality of spinal rods 50, which may be relatively disposed in a side by side, irregular, uniform, non-uniform, offset and/or staggered orientation or arrangement.

In some embodiments, spinal rod 50 can have a uniform thickness/diameter. In some embodiments, spinal rod 50 may have various surface configurations, such as, for example, rough, threaded for connection with surgical instruments, arcuate, undulating, dimpled, polished and/or textured. In some embodiments, the thickness defined by spinal rod 50 may be uniformly increasing or decreasing, or have alternate diameter dimensions along its length. In some embodiments, spinal rod 50 may have various cross section configurations, such as, for example, oval, oblong, triangular, rectangular, square, polygonal, irregular, uniform, non-uniform, variable and/or tapered. In some embodiments, spinal rod 50 may have various lengths.

In assembly, operation and use, a surgical system including spinal correction system 10, similar to the systems and methods described herein, is employed with a surgical procedure, such as, for example, a correction treatment of an affected portion of a spine, for example, a correction treatment to treat adolescent idiopathic scoliosis and/or Scheuermann's kyphosis of a spine. In some embodiments, one or all of the components of spinal correction system 10 can be delivered or implanted as a pre-assembled device or can be assembled in situ. Spinal correction system 10 may be completely or partially revised, removed or replaced.

For example, as shown in FIGS. 6 and 7, spinal correction system 10 can be employed with a surgical correction treatment of an applicable condition or injury of an affected section of a spinal column and adjacent areas within a body, such as, for example, at least a vertebra V1 and a vertebra V2 of vertebrae V. In some embodiments, spinal correction system 10 may be employed with one or a plurality of vertebrae.

In use, to treat a selected section of vertebrae V, a medical practitioner obtains access to a surgical site including vertebrae V in any appropriate manner, such as through incision and retraction of tissues. In some embodiments, spinal correction system 10 can be used in any existing surgical method or technique including open surgery, mini-open surgery, minimally invasive surgery and percutaneous surgical implantation, whereby vertebrae V is accessed through a mini-incision, or a sleeve that provides a protected passageway to the area. Once access to the surgical site is obtained, the particular surgical procedure can be performed for treating the spine disorder,

An incision is made in the body of a patient and a cutting instrument (not shown) creates a surgical pathway for implantation of components of spinal correction system 10. A preparation instrument (not shown) can be employed to prepare tissue surfaces of vertebrae V, as well as for aspiration and irrigation of a surgical region.

Spinal rod 50 is delivered along the surgical pathway to the surgical site adjacent a lateral side of vertebrae V. Tether 12 is delivered along the surgical pathway to the surgical site adjacent the lateral side of vertebrae V1, V2, Crimp 30 is pre-assembled with tether 12 in an initial position such that tether 12 is loose, slack or includes a lower degree of tension. In some embodiments, tether 12 is connected with crimp 30 in situ. In some embodiments, spinal correction system 10 includes a plurality of tethers 12 that are delivered adjacent vertebrae V and spaced apart along spinal rod 50, each tether 12 being positioned for connection of individual vertebra with spinal rod 50, as described herein.

Lead 20 of each tether 12 is connected with vertebra V2 and guided through a sub-laminar cavity to resist and/or prevent non-desirable and/or harmful engagement with laminae L of vertebrae, for example, vertebra V2 and/or dura matter D and without adhering to dura matter D and/or surfaces of laminae L. Lead 20 is passed around laminae L of vertebra V2, spinal rod 50 and through opening 38. Tether 12 forms loop 24 about vertebra V2 and spinal rod 50, as shown in FIG. 6.

Lead 20 is drawn or tensioned and crimp 30 is manipulated and/or engaged with a surgical instrument (not shown) to abut spinal rod 50 for translating spinal rod 50 to a selected location, such as, for example, the surgical site adjacent the lateral side of vertebrae V1, V2. Crimp 30 is translatable in a first direction and/or in a second opposing direction, as described herein, relative to tether 12 such that crimp 30 translates along tether 12 to drive surface 34 into engagement with spinal rod 50. As crimp 30 translates along tether 12 and drives spinal rod 50 to the surgical site and adjacent vertebra V2, a tensioning force increases in tether 12 causing spinal rod 50 to move adjacent vertebra V2.

Crimp 30 is translated relative to tether 12 to adjustably tighten tether 12 with spinal rod 50 and vertebra V2 between the initial position, as shown in FIG. 6, and a second position such that surface 34 abuts spinal rod 50 to apply force to spinal rod 50 for fixation with vertebra V2, as shown in FIG. 7. In the second position, crimp 30 is deformed, as described herein, via surgical instrument or manipulation, into engagement with tether 12 such that surface 32 is fixed with surface 26 to prevent translation of crimp 30 relative to tether 12. Crimp 30 fixes spinal rod 50 with the lateral side of vertebrae V and tether 12 is taught and includes a greater degree of tension relative to the initial position. Spinal correction system 10 stabilizes vertebrae V and affects growth for a correction treatment to treat spine pathologies, as described herein.

In some embodiments, spinal correction system 10 includes a second spinal rod (not shown) delivered along the surgical pathway to the surgical site adjacent a contra-lateral side of vertebrae V. The second spinal rod is connected with the contra-lateral side of vertebrae V via one or more tethers 12, similar to spinal rod 50 described herein. Spinal rod 50 and the second spinal rod are fixed with vertebrae V in a side by side orientation and/or bi-lateral arrangement to stabilize vertebrae V and affect growth for a correction treatment to treat spine pathologies, as described herein.

Upon completion of the procedure, the surgical instruments, assemblies and non-implanted components of spinal correction system 10 are removed from the surgical site and the incision is closed. One or more of the components of surgical implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal correction system 10.

In some embodiments, spinal correction system 10 includes an agent, which may be disposed, packed, coated or layered within, on or about the components and/or surfaces of spinal correction system 10. In some embodiments, the agent may include bone growth promoting material, such as, for example, bone graft to enhance fixation of the bone fasteners with vertebrae. In some embodiments, the agent may include one or a plurality of therapeutic agents and/or pharmacological agents for release, including sustained release, to treat, for example, pain, inflammation and degeneration.

In some embodiments, the components of spinal correction system 10 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of spinal correction system 10 may be used to prevent or minimize curve progression in individuals of various ages.

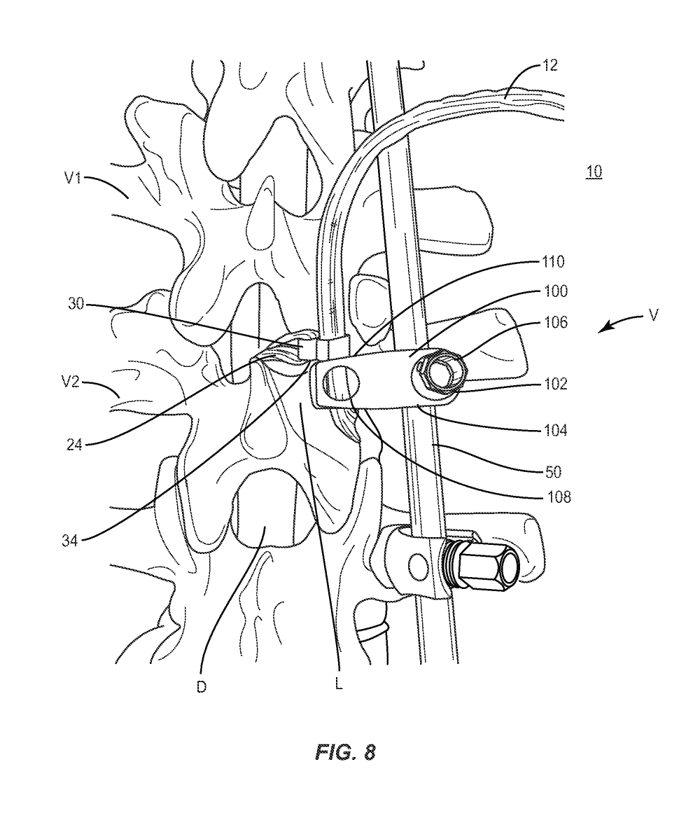

In one embodiment, as shown in FIG. 8, spinal correction system 10, similar to the systems and methods described herein, comprises a connector 100 that is configured to connect spinal rod 50 with tether 12 to secure spinal rod 50 with laminae L, similar to that described herein. Connector 100 includes a surface 102 that defines an opening 104 configured for disposal of spinal rod 50. Spinal rod 50 is disposable with opening 104 and may be selectively translated within opening 104 relative to connector 100 and/or vertebrae. In some embodiments, spinal rod 50 is fixed with connector 100 with a locking element, such as, for example, as set screw 106. Surface 102 defines an opening 108 configured for disposal of tether 12. Connector 100 includes a surface 110 configured for engagement with surface 34 of crimp 30 to secure tether 12 and/or spinal rod 50 with vertebrae, similar to that described herein.

In use, similar to that described with regard to FIGS. 6 and 7, to treat a selected section of vertebrae V, a spinal rod 50 and a connector 100 are delivered along the surgical pathway to the surgical site. Spinal rod 50 is selectively disposed with opening 104 for alignment with vertebrae V1, V2. Tether 12 is delivered along the surgical pathway to the surgical site adjacent the lateral side of vertebrae V1, V2. Crimp 30 is pm-assembled with tether 12 in an initial position such that tether 12 is loose, slack or includes a lower degree of tension.

Lead 20 of tether 12 is connected with vertebra V2 and guided through a sub-laminar cavity to resist and/or prevent non-desirable and/or harmful engagement with laminae L of vertebrae, for example, vertebra V2 and/or dura matter D and without adhering to dura matter D and/or surfaces of laminae L. Lead 20 is passed around laminae L of vertebra V2, through opening 108 and through opening 38. Tether 12 forms loop 24 about vertebra V2, as shown in FIG. 8.

Lead 20 is drawn or tensioned and crimp 30 is manipulated and/or engaged with a surgical instrument (not shown) to abut connector 100 for securing tether 12 to connector 100 to a selected location, such as, for example, the surgical site adjacent the lateral side of vertebrae V1, V2. Crimp 30 is translatable in a first direction and/or in a second opposing direction, as described herein, relative to tether 12 such that crimp 30 translates along tether 12 to position surface 34 into engagement with connector 100 to secure tether 12 with connector 100. As crimp 30 translates along tether 12, a tensioning force increases in tether 12 securing connector 100 and spinal rod 50 with tether 12 adjacent vertebra V2.

In the second position, crimp 30 is deformed, as described herein, via surgical instrument or manipulation, into engagement with tether 12 such that surface 32 is fixed with surface 26 to prevent translation of crimp 30 relative to tether 12. Crimp 30 fixes tether 12, connector 100 and spinal rod 50 with the lateral side of vertebrae V and tether 12 is taught and includes a greater degree of tension relative to the initial position. Spinal correction system 10 including connector 100 stabilizes vertebrae V and affects growth for a correction treatment to treat spine pathologies, as described herein

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
a longitudinal member extending between a first end and a second end; and
a reduction member connected with the second end and defining at least one opening configured for disposal of the longitudinal member, the reduction member including an outer surface engageable with a spinal implant and being translatable relative to the longitudinal member.

2. A spinal construct as recited in claim 1, wherein the outer surface abuts the spinal implant such that the reduction member translates the spinal implant adjacent spinal tissue.

3. A spinal construct as recited in claim 1, wherein the reduction member is translatable between a first position and a second position such that the longitudinal member includes a tension and the spinal implant is fixed with spinal tissue.

4. A spinal construct as recited in claim 1, wherein the reduction member comprises a crimp that resists movement in at least one direction.

5. A spinal construct as recited in claim 4, wherein the crimp includes an inner surface having at least one penetrating element engageable with the longitudinal member to resist movement in the at least one direction.

6. A spinal construct as recited in claim 1, wherein the at least one opening includes a first opening and a second opening, the openings being separated by a wall.

7. A spinal construct as recited in claim 1, wherein the longitudinal member comprises a loop encircled about the spinal implant.

8. A spinal construct as recited in claim 1, wherein the first end comprises a pliable lead.

9. A spinal construct as recited in claim 8, wherein the pliable lead includes a paddle configuration having a blunt tip.

10. A spinal construct as recited in claim 8, wherein the pliable lead connects the first end and the second end.

11. A spinal construct as recited in claim 1, wherein the spinal implant includes a connector configured for disposal of a spinal rod.

12. A spinal construct as recited in claim 1, wherein the spinal implant comprises a spinal rod configured to extend over a plurality of vertebral levels.

13. A spinal construct comprising:
a flexible tether extending between a first end and a second end; and
a reduction member connected with the second end and defining at least one opening configured for disposal of the tether, the reduction member disposed to abut a spinal implant for translation relative to the tether.

14. A spinal construct as recited in claim 13, wherein the reduction member is translatable between a first position and a second position such that the tether includes a tension and the spinal implant is fixed with spinal tissue.

15. A spinal construct as recited in claim 13, wherein the reduction member comprises a crimp that resists movement in at least one direction.

16. A spinal construct as recited in claim 15, wherein the crimp includes an inner surface having at least one penetrating element engageable with the tether to resist movement in the at least one direction.

17. A method for treating a spine, the method comprising the steps of:
providing a tether including a reduction member;
connecting the tether with spinal tissue;
providing a spinal rod;
translating the reduction member relative to the tether such that an outer surface of the reduction member engages the spinal rod to dispose the spinal rod adjacent the spinal tissue.

18. A method for treating a spine as recited in claim 17, wherein the step of translating includes the reduction member being movable in a first direction relative to the tether to translate the spinal rod adjacent the spinal tissue and prevented from movement in a second direction.

19. A method for treating a spine as recited in claim 17, wherein the step of translating includes the reduction member being movable between a first position and a second position such that the tether includes a tension and the spinal rod is fixed with spinal tissue.

20. A method for treating a spine as recited in claim 17, further comprising the step of engaging the reduction member to fix the spinal rod with spinal tissue.
